(19) United States
(12) Patent Application Publication
(10) Pub. No.: US 2023/0173046 A1
(43) Pub. Date: Jun. 8, 2023

(54) MULTICOMPONENT CHEMICAL COMPOSITION OF A PEPTIDE-BASED NEOANTIGEN VACCINE

(71) Applicant: AMAZON TECHNOLOGIES, INC., RENO, NV (US)

(72) Inventors: Frank Wilhelm SCHMITZ, Seattle, WA (US); David HECKERMAN, Bellevue, WA (US); Layne Christopher PRICE, Seattle, WA (US); Antje HEIT, Seattle, WA (US)

(21) Appl. No.: 17/788,651

(22) PCT Filed: May 19, 2022
(86) PCT No.: PCT/US2022/030037

§ 371 Date: Jun. 23, 2022

Related U.S. Application Data
(60) Provisional application No. 63/194,041, filed on May 27, 2021.

(51) Int. Cl. A61K 39/00; A61K 39/39; A61K 45/06

(52) U.S. Cl. CPC A61K 39/0011; A61K 39/39; A61K 45/06; A61K 2039/545; A61K 2039/55516

(57) ABSTRACT

Provided herein are immunogenic compositions comprising tumor-specific neoantigen long peptides, tumor-specific neoantigen short peptides, and adjuvant, optionally a helper peptide, and optionally a tumor-specific peptide. The disclosure also provides methods of using these immunogenic compositions for treating cancer.

### 1. CROSS-REFERENCE TO RELATED APPLICATIONS

The present application claims the benefit of U.S. Provisional Pat. Application 63/194,041, filed May 27, 2021, which is incorporated herein by reference in its entirety.

### 2. REFERENCE TO A SEQUENCE LISTING

This application contains a Sequence Listing in computer readable form. The computer readable form is incorporated herein by reference. Said ASCII copy, created on May 3, 2022, is named 146401_091808_SL.txt and is 1,108 bytes in size.

### 3. BACKGROUND

Cancer is a leading cause of death worldwide accounting for 1 in 4 of all deaths. Siegel et al., CA: A Cancer Journal for Clinicians, 68:7-30 (2018). There were 18.1 million new cancer cases and 9.6 million cancer-related deaths in 2018. Bray et al., CA: A Cancer Journal for Clinicians, 68(6):394-424. There are a number of existing standard of care cancer therapies, including ablation techniques (e.g., surgical procedures and radiation) and chemical techniques (e.g., chemotherapeutic agents). Unfortunately, such therapies are frequently associated with serious risk, toxic side effects, and extremely high costs, as well as uncertain efficacy.

Cancer immunotherapy (e.g., cancer vaccine) has emerged as a promising cancer treatment modality. The goal of cancer immunotherapy is to harness the immune system for selective destruction of cancer while leaving normal tissues unharmed. Traditional cancer vaccines typically target tumor-associated antigens. Tumor-associated antigens are typically present in normal tissues, but overexpressed in cancer. However, because these antigens are often present in normal tissues immune tolerance can prevent immune activation. Several clinical trials targeting tumor-associated antigens have failed to demonstrate a durable beneficial effect compared to standard of care treatment. Li et al., Ann Oncol., 28 (Suppl 12): xii11—xii17 (2017).

Neoantigens represent an attractive target for cancer immunotherapies. Neoantigens are non-autologous proteins with individual specificity. Neoantigens are derived from random somatic mutations in the tumor cell genome and are not expressed on the surface of normal cells. Id. Because neoantigens are expressed exclusively on tumor cells, and thus do not induce central immune tolerance, cancer vaccines targeting cancer neoantigens have potential advantages, including decreased central immune tolerance and improved safety profile. Id.

The mutational landscape of cancer is complex and tumor mutations are generally unique to each individual subject. Most somatic mutations detected by sequencing do not result in effective neoantigens. Only a small percentage of mutations in the tumor DNA, or a tumor cell, are transcribed, translated, and processed into a tumor-specific neoantigen with sufficient accuracy to design a vaccine that is likely to be effective. Further, not all neoantigens are immunogenic. In fact, the proportion of T cells spontaneously recognizing endogenous neoantigens is about 1% to 2%. See, Karpanen et al., Front Immunol., 8:1718 (2017). Moreover, the cost and time associated with the manufacture of neoantigen vaccines is significant.

Thus, significant challenges remain for developing personalized cancer vaccines comprising neoantigens.

### 4. SUMMARY

The invention relates to personalized (i.e., subject-specific) immunogenic compositions (e.g., a cancer vaccine) comprising a unique combination of components. The immunogenic compositions described herein comprise a plurality of tumor-specific neoantigen long peptides, a plurality of tumor-specific neoantigen short peptides, and an adjuvant. The immunogenic composition may optionally comprise a helper peptide. The immunogenic composition may optionally comprise a tumor-specific frameshift peptide.

The immunogenic composition may comprise up to about 50 tumor-specific neoantigen long peptides and/or short peptides. The immunogenic composition may comprise about 10 to about 20 tumor-specific neoantigen long peptides and/or short peptides. It is preferred that the immunogenic composition comprises about 19 tumor-specific neoantigen long peptides and/or short peptides.

The immunogenic composition may comprise at least about 2 or more tumor-specific neoantigen long peptides. The immunogenic composition may comprise about 2 to about 18 tumor-specific neoantigen long peptides. The immunogenic composition can typically comprise at least about 10 to about 15 tumor-specific neoantigen long peptides. The immunogenic composition may comprise at least about 2 or more tumor-specific neoantigen short peptides. The immunogenic composition may comprise at least about 2 to about 10 tumor-specific neoantigen short peptides.

Typically, each of the tumor-specific neoantigen long peptides in the immunogenic composition are different. Typically, each of the tumor-specific short peptides in the immunogenic composition are different.

The immunogenic composition may comprise two or more tumor-specific frameshift peptides. The tumor-specific neoantigen long peptides and/or short peptides can be divided into two or more peptide pools. Preferably, the tumor-specific neoantigen long peptides and/or short peptides are divided into about four peptide pools. Generally, each peptide pool can comprise about 5 or less tumor-specific neoantigen long peptides and/or short peptides. The one or more peptide pools can optionally comprise a helper peptide. The one or more peptide pools can comprise one or more tumor-specific frameshift peptides.

As an example, three peptide pools may comprise about 5 tumor-specific neoantigen long peptides and/or short peptides and one peptide pool may comprise 4 tumor-specific neoantigen long peptides and/or short peptides and a helper peptide. Each peptide pool may comprise different tumor-specific neoantigen long peptides and/or short peptides. Tumor-specific neoantigen long peptides may be about 15 to about 30 amino acids in length. Tumor-specific neoantigen short peptides may be about 5 to about 15 amino acids in length.

The adjuvant used in the immunogenic composition may be a Toll-like receptor agonist, a NOD-like receptor agonist, an Mda5 agonist, a RIG-I, PKR agonist, a STING agonist, or other innate immune sensing pathway agonists. Each of the peptide pools may comprise the adjuvant.

The helper peptide may be a pan-DR helper epitope (PADRE), a tetanus helper peptide, a hepatitis B surface antigen helper T-cell epitope, a pertussis toxin helper T-cell epitope, a measles virus F protein helper T-cell epitope, a Chlamydia trachomitis major outer membrane protein helper T-cell epitope, a diphtheria toxin helper T-cell epitope, a Plasmodium falciparum circumsporozoite helper T-cell epitope, a Schistosoma mansoni triose phosphate isomerase helper T-cell epitope, a keyhole limpet hemocyanin, a Plasmodium vivax B cell epitope (PVB), a Escherichia coli TraT helper T-cell epitope, a synthetic T helper epitope, immune-enhancing analogs and segments of any of the foregoing helper peptides. Preferably, the helper peptide is a pan-DR helper epitope (PADRE).

The immunogenic composition disclosed herein can induce a polyfunctional CD4⁺ and CD8⁺ response.

The disclosure also relates to pharmaceutical compositions comprising the immunogenic composition disclosed herein.

This disclosure also relates to methods of treating cancer in a subject in need thereof comprising administering the personalized immunogenic composition described herein. The methods disclosed herein can be suited for treating any number of cancers. The tumor can be from melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Preferably, the cancer is melanoma, breast cancer, lung cancer, colon cancer and urothelial cancer. The subject suitable for the methods disclosed herein can be diagnosed with cancer, is already suffering from cancer, has recurrent cancer, or is at risk of developing cancer.

The subject can be administered at least one or more doses of the immunogenic composition disclosed herein. Typically, the subject is administered at least six doses of the immunogenic composition at different times.

The immunogenic composition can be administered as one or more peptide pools at each dose. The subject can be administered six doses of the two or more peptide pool at different times. The subject can be administered each peptide pool in one to four extremities of the subject. The peptide pool can be administered at different locations. The subject can be administered each peptide pool on different extremities of the subject. The subject can be administered each peptide pool on the same extremity of the subject at each administration. Each dose of the immunogenic composition can be administered at least about 1 week to about 4 weeks after administration of the prior dose of the immunogenic composition.

An adjuvant can be administered between each dose of the immunogenic composition. The adjuvant can be administered weekly between each dose of the immunogenic composition. The adjuvant may be a Toll-like receptor agonist, a NOD-like receptor agonist, an Mda5 agonist, a RIG-I, PKR agonist, a STING agonist, or other innate immune sensing pathway agonists.

The methods disclosed herein can further comprise administering at least one or one checkpoint inhibitors. The checkpoint inhibitor can be an inhibitor of the programmed death-1 (PD-1) pathway, Lag3 pathway, Tim3 pathway, ICOS pathway, OX-40, GITR pathway, or 4-1BB pathway. In particular, the inhibitor of the PD-1 pathway can be an anti-PD-1 antibody, small molecule, peptide, or inhibit the pathway by genetic means (e.g. short interfering RNA or CRISPR-mediated gene editing). A checkpoint inhibitor of interest may be an anti-cytotoxic T-lymphocyte-associated antigen 4 (CTLA4) antibody, small molecule, peptide, or inhibit the pathway by genetic means (e.g. short interfering RNA or CRISPR-mediated gene editing).

The immunogenic composition may be administered subcutaneous, intramuscular, transcutaneous, intradermal, transdermal, intravenous, intra-tumoral, into a lymph node, or intraperitoneal administration. It is preferred that the immunogenic composition disclosed herein is administered by intramuscular administration.

### 5. BRIEF DESCRIPTION OF THE DRAWINGS

FIG. 1 is a schematic showing the composition of the vaccine peptide pools.

FIG. 2 is a schematic showing the treatment regimen of the immunogenic composition.

### 6. DETAILED DESCRIPTION

This disclosure relates to potent personalized cancer immunogenic compositions (e.g., subject-specific immunogenic compositions) comprising a unique combination of components. The immunogenic compositions described herein comprise a plurality of tumor-specific neoantigen long peptides, a plurality of tumor-specific neoantigen short peptides, and an adjuvant. The immunogenic composition may optionally comprise a helper peptide. The immunogenic composition may optionally comprise a tumor-specific frameshift peptide. The immunogenic composition can comprise up to about 50 tumor-specific neoantigen long peptides and/or short peptides. Typically, the immunogenic composition comprises about 10 to about 20 tumor-specific neoantigen long peptides and/or short peptides. Preferably, the immunogenic composition comprises about 19 tumor-specific neoantigen long peptides and/or short peptides. The immunogenic composition can be divided into two or more peptide pools that comprise the tumor-specific neoantigen long peptides and/or short peptides and the adjuvant. The one or more peptide pools can further comprise a helper peptide. The one or more peptide pools may further comprise a tumor-specific frameshift peptide. It is preferred that the immunogenic composition is divided into about 4 peptide pools each with about 5 or less peptides (i.e., tumor-specific neoantigen long peptides or short peptides, a helper peptide, or a tumor-specific frameshift peptide). Each peptide pool can also comprise the adjuvant.

The disclosure also relates to methods of treating cancer in a subject in need thereof by administering an immunogenic composition comprising tumor-specific neoantigen peptides. The subject can be administered at least one or more doses of the immunogenic composition. Typically, the subject can be administered about six doses of the immunogenic composition at different times. The immunogenic composition can be administered as one or more peptides at each dose. The peptide pools can be administered in different extremities (e.g., about one to about four). The peptide pool can be administered at the same extremity of the subject at each administration.

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “cancer” refers to the physiological condition in subjects in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer, Hormone receptor positive breast cancer), osteosarcoma, melanoma, colon cancer, colorectal cancer, endometrial (e.g., serous) or uterine cancer, salivary gland carcinoma, kidney cancer, liver cancer, prostate cancer, vulvar cancer, thyroid cancer, hepatic carcinoma, and various types of head, neck cancers, and brain cancers. Triple negative breast cancer refers to breast cancer that is negative for expression of the genes for estrogen receptor (ER), progesterone receptor (PR), and Her2/neu. Hormone receptor positive breast cancer refers to breast cancer that is positive for at least one of the following: ER or PR, and negative for Her2/neu (HER2).

The term “helper peptide” as used herein refers to a foreign peptide that functions as a nonspecific vaccine helper epitope and induces an increased immune response by activating CD4 T-cells.

The term “frameshift mutation” as used herein refers to a change in the nucleic acid sequence within the open reading frame encoding the protein, which results in a change in the downstream reading frame of the mutation, thereby producing a protein with a changed sequence compared to the wild-type protein. Typically, frameshift mutations are caused by indels (i.e., insertions or deletions of one or more nucleotides) that is not a multiple of three.

The term “tumor-specific frameshift peptide” as used herein refers to a tumor-specific neoantigen peptide that contains a frameshift mutation.

The term “neoantigen” as used herein refers to an antigen that has at least one alteration that makes it distinct from the corresponding parent antigen, e.g., via mutation in a tumor cell or post-translational modification specific to a tumor cell. A mutation can include a frameshift, indel, missense or nonsense substitution, splice site alteration, genomic rearrangement or gene fusion, or any genomic expression alteration giving rise to a neoantigen. A mutation can include a splice mutation. Post-translational modifications specific to a tumor cell can include aberrant phosphorylation. Post-translational modifications specific to a tumor cell can also include a proteasome-generated spliced antigen. See, Lipe et al., Science, 354(6310):354:358 (2016). In general, point mutations account for about 95% mutations in tumors and indels and frame-shift mutations account for the rest. See, Snyder et al., N Engl J Med., 371:2189-2199 (2014).

As used herein the term “tumor-specific neoantigen” is a neoantigen present in a subject’s tumor cell or tissue, but not in the subject’s normal cell or tissue.

The term “subject” as used herein refers to any animal, such as any mammal, including but not limited to, humans, non-human primates, rodents, and the like. In some embodiments, the mammal is a mouse. In some embodiments, the mammal is a human.

Additional description of the methods and guidance for the practice of the methods are provided herein.

### A. Immunogenic Composition

### a. Neoantigen Peptides

The immunogenic composition can be formulated so that the selection and number of tumor-specific neoantigens is tailored to the subject’s particular cancer. For example, the selection of the tumor-specific neoantigens can be dependent on the specific type of cancer, the status of the cancer, the immune status of the subject, and the MHC-type of the subject.

The immunogenic composition can comprise at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50 or more tumor-specific neoantigen peptides (e.g., tumor-specific neoantigen long peptides and/or short peptides). The immunogenic composition can comprise up to about 100 tumor-specific. The immunogenic composition can contain about 10-20 tumor-specific neoantigens, about 10-30 tumor-specific neoantigens, about 10-40 tumor-specific neoantigens, about 10-50 tumor-specific neoantigens, about 10-60 tumor-specific neoantigens, about 10-70 tumor-specific neoantigens, about 10-80 tumor-specific neoantigens, about 10-90 tumor-specific neoantigens, or about 10-100 tumor-specific neoantigens. Typically, the immunogenic composition comprises at least about 10 tumor-specific neoantigens. The immunogenic composition disclosed herein preferably comprises 10 to about 20 tumor-specific neoantigens. For example, the immunogenic composition can comprise about 10, about 11, about 12, about 13, about 14, about 15, about 16, about 17, about 18, about 19, or about 20 tumor-specific neoantigens. Preferably, the immunogenic composition can comprise about 19 tumor-specific neoantigens. Preferably, the immunogenic composition can comprise about 20 tumor-specific neoantigens. Each of the tumor-specific neoantigens in the immunogenic composition are preferably different.

The immunogenic composition disclosed herein comprises a plurality of tumor-specific neoantigen long peptides and a plurality of tumor-specific neoantigen short peptides. Tumor-specific neoantigen long peptides are internalized by antigen-presenting cells and processed for MCH presentation. MHC class II molecules typically bind to peptides that are longer in length. MHC class II can accommodate peptides which are generally about 13 amino acids in length to about 25 amino acids in length. In embodiments, the one or more tumor-specific neoantigens are long peptides about 13 to 25 amino acids in length. MHC class I molecules typically bind to short peptides. Tumor-specific neoantigen short peptides bind directly to MHC molecules. MHC class I molecules can bind to short peptides. MHC class I molecules can accommodate peptides generally about 8 amino acids to about 10 amino acids in length.

The immunogenic composition can comprise at least about 2 or more tumor-specific neoantigen long peptides. The immunogenic composition can comprise at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50 or more tumor-specific neoantigen long peptides. The immunogenic composition can contain about 2 to 20 tumor-specific neoantigen long peptides, about 10 to 20 tumor-specific neoantigen long peptides, about 10 to 30 tumor-specific neoantigen long peptides, about 10 to 40 tumor-specific neoantigen long peptides, or about 10 to 50 tumor-specific neoantigen long peptides. Typically, the immunogenic composition comprises at least about 10 tumor-specific neoantigen long peptides.

The immunogenic composition disclosed herein preferably comprises 10 to about 15 tumor-specific neoantigen long peptides. For example, the immunogenic composition can comprise about 10, about 11, about 12, about 13, about 14, about 15, about 16, about 17, or about 18 tumor-specific neoantigen long peptides. In general, the immunogenic composition disclosed herein comprises more tumor-specific neoantigen long peptides relative to tumor-specific neoantigen short peptides. Each of the tumor-specific long peptides in the immunogenic composition are preferably different.

The immunogenic composition can comprise at least about 2 or more tumor-specific neoantigen short peptides. The immunogenic composition can comprise at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50 or more tumor-specific neoantigen short peptides. The immunogenic composition can contain about 2 to about 10 tumor-specific neoantigen short peptides. For example, the immunogenic composition can comprise at least about 2, about 3, about 4, about 5, about 6, about 7, about 8, about 9, or about 10 tumor-specific neoantigen short peptides.

The immunogenic composition can contain about 2 to 15 tumor-specific neoantigen short peptides, about 2 to 10 tumor-specific neoantigen short peptides, about 4 to 10 tumor-specific neoantigen short peptides, about 5 to 10 tumor-specific neoantigen short peptides, about 6 to 10 tumor-specific neoantigen short peptides, about 7 to 10 tumor-specific neoantigen short peptides, or about 8 to 10 tumor-specific neoantigen short peptides. The immunogenic composition disclosed herein preferably comprises 2 to about 10 tumor-specific neoantigen short peptides. For example, the immunogenic composition can comprise about 2, about 3, about 4, about 5, about 6, about 7, about 8, about 9, or about 10, tumor-specific neoantigen short peptides. In general, the immunogenic composition disclosed herein comprises less tumor-specific neoantigen short peptides relative to tumor-specific neoantigen long peptides. Each of the tumor-specific short peptides in the immunogenic composition are preferably different.

The tumor-specific neoantigen long peptides can be about 15 to about 30 amino acids in length. The tumor-specific neoantigen long peptides can be about 15 amino acids in length, about 16 amino acids in length, about 17 amino acids in length, about 18 amino acids in length, about 19 amino acids in length, about 20 amino acids in length, about 21 amino acids in length, about 22 amino acids in length, about 23 amino acids in length, about 24, amino acids in length, about 25 amino acids in length, about 26 amino acids in length, about 27 amino acids in length, about 28 amino acids in length, about 29 amino acids in length, or about 30 amino acids in length.

The tumor-specific neoantigen short peptides can be about 5 amino acids in length to about 15 amino acids in length. The tumor-specific neoantigen short peptides can be about 5 amino acids in length, about 6 amino acids in length, about 7 amino acids in length, about 8 amino acids in length, about 9 amino acids in length, about 10 amino acids in length, about 11 amino acids in length, about 12 amino acids in length, about 13 amino acids in length, about 14 amino acids in length, or about 15 amino acids in length.

Whether long or short, the tumor-specific neoantigen peptides can arise from any mechanism leading to transcripts or translated peptides unique to the tumor. Examples of such mechanisms include, but are not limited to, insertions, deletions, and/or rearrangements of tumor DNA; errors in transcription; altered and/or incomplete intron splicing of primary transcripts; or errors in translation.

### b. Peptide Pools

The tumor-specific neoantigen long peptides and/or short peptides of the immunogenic composition can be divided into two or more peptide pools. For instance, the tumor-specific neoantigen long peptides and/or short peptides of the immunogenic composition can be divided into about 2, about 3, about 4, about 5, about 6, about 7, about 8, about 9, about 10, about 11, about 12, about 13, about 14, about 15, about 16, about 17, about 18, about 19, about 20, or more peptide pools. The desired number of peptide pools can be determined by the number of tumor-specific neoantigen long peptides and/or short peptides in the immunogenic composition. For example, an immunogenic composition comprising about 40 tumor-specific neoantigen long peptides and/or short peptides may be divided into about 5 to about 10 peptide pools. For example, an immunogenic composition comprising about 30 tumor-specific neoantigen long peptides and/or short peptides may be divided into about 5 to about 8 peptide pools. For example, an immunogenic composition comprising about 20 tumor-specific neoantigen long peptides and/or short peptides may be divided into about 2 to about 5 peptide pools.

In the preferred immunogenic composition described herein, the tumor-specific neoantigen long peptides and/or short peptides may be divided into about 2 to about 5 peptide pools. Without being bound by theory, it is believed that dividing the tumor-specific neoantigen long peptides and/or short peptides can facilitate co-solubility of the immunogenic composition. Preferably, the tumor-specific neoantigen long peptides and/or short peptides can be divided into about 4 peptide pools or about 5 peptide pools.

Each peptide pool disclosed herein may comprise up to about 20 tumor-specific neoantigen long peptide and/or short peptides. For example, each peptide pool disclosed herein may comprise about 1, about 2, about 3, about 4, about 5, about 6, about 7, about 8, about 9, about 10, about 11, about 12, about 13, about 14, about 15, about 16, about 17, about 18, about 19, or about 20 tumor-specific neoantigen long peptide and/or short peptides. It is preferred that each peptide pool comprises about 5 peptide pools or less. For instance, each peptide pool may comprise about 1, about 2, about 3, about 4, or about 5 tumor-specific neoantigen long peptide and/or short peptides.

Each peptide pool may comprise the same number of tumor-specific neoantigen long peptide and/or short peptides. For example, each peptide pool may comprise about 1, about 2, about 3, about 4, or about 5 tumor-specific neoantigen long peptide and/or short peptides.

Each peptide pool may comprise a different number tumor-specific neoantigen long peptide and/or short peptides. For example, 4 peptide pools may comprise 5 tumor-specific neoantigen long peptide and/or short peptides and 1 peptide pool may comprise 4 tumor-specific neoantigen long peptide and/or short peptides. For example, 3 peptide pools may comprise 5 tumor-specific neoantigen long peptide and/or short peptides, 1 peptide pool may comprise 4 tumor-specific neoantigen long peptide and/or short peptides, and 1 peptide pool may comprise 3 tumor-specific neoantigen long peptide and/or short peptides. For example, 2 peptide pools may comprise 5 tumor-specific neoantigen long peptide and/or short peptides, 1 peptide pool may comprise 4 tumor-specific neoantigen long peptide and/or short peptides, 1 peptide pool may comprise 3 tumor-specific neoantigen long peptide and/or short peptides, peptide pool may comprise tumor-specific neoantigen long peptide and/or short peptides.

The immunogenic composition disclosed herein may also comprise at least one helper peptide. In some instances, one peptide pool of the immunogenic composition may comprise a helper peptide. In some instances, one or more peptide pools disclosed herein can comprise a helper peptide. A peptide pool may comprise about 1, about 2, about 3, about 4, about 5 or more helper peptides. Although, generally a peptide pool preferably comprises a single helper peptide. As an example, when the immunogenic composition is divided into 5 peptide pools, a single peptide pool may comprise a helper peptide.

The immunogenic composition further comprises an adjuvant. One or more peptide pool may comprise an adjuvant. In some instances, each peptide pool may comprise an adjuvant. In other instances, the adjuvant may be present in a portion of the peptide pools. For example, when the immunogenic composition is divided into 5 peptide pools, about 1, about 2, about 3, about 4, or about 5 peptide pools can comprise an adjuvant. Preferably, each peptide pool comprises an adjuvant.

The immunogenic composition disclosed herein may also comprise at least one or more tumor-specific frameshift peptides. The immunogenic composition may comprise about 1, about 2, about 3, about 4, about 5, about 6, about, about 7, about 8, about 9, about 10, about 11, about 12, about 13, about 14, about 15 or more tumor-specific frameshift peptides. In some instances, one or more peptide pools may comprise a tumor-specific frameshift peptide. A peptide pool may comprise about 1, about 2, about 3, about 4, about 5 or more tumor-specific frameshift peptides.

The immune response in the subject can include presentation of one or more tumor-specific neoantigens to the tumor cell surface, presentation of one or more tumor-specific neoantigens by one or more MHC molecules on the tumor cell, or that one or more tumor-specific neoantigens is capable of presentation to T cells by antigen presenting cells.

The immune response in the subject can be a CD4+ mediated response, a CD8+ mediated response or a polyfunctional CD4+ and mediated response.

The immunogenic composition can contain individualized components, according to their personal needs of the particular subject.

### c. Helper Peptide

The immunogenic composition disclosed herein may also comprise at least one helper peptide. The helper peptide can be any suitable peptide that stimulates CD4+ T cell responses that are not specific for cancer antigens, but may stimulate recall responses or other nonspecific help. Activation of CD4 T-cells can support CD8+ T-cell.

Helper peptides are a sequence of amino acids (natural or non-natural amino acids) that have T-cell helper activity. Helper peptides are recognized by T-helper lymphocytes, which play an important role in establishing and maximizing the capabilities of the immune system, and are involved in activating and directing other immune cells, such as for example cytotoxic T lymphocytes.

A helper peptide can comprise a continuous or discontinuous epitope. Helper peptides, including analogs and segments of helper peptides, are capable of enhancing or stimulating an immune response. The helper peptide may be from about 10 to about 150 amino acids in length, and more particularly about 10 to about 50 amino acids in length. When multiple helper peptides are present, each helper peptides typically acts independently.

The helper peptide is generally not a tumor-specific neoantigen.

Helper peptides which may be used in the immunogenic compositions disclosed herein include, for example, hepatitis B surface antigen helper T-cell epitopes, pertussis toxin helper T-cell epitopes, measles virus F protein helper T-cell epitope, Chlamydia trachomitis major outer membrane protein helper T-cell epitope, diphtheria toxin helper T-cell epitopes, Plasmodium falciparum circumsporozoite helper T-cell epitopes, Schistosoma mansoni triose phosphate isomerase helper T-cell epitopes, keyhole limpet hemocyanin, Plasmodium vivax B cell epitopes (PVB), Escherichia coli TraT helper T-cell epitopes and immune-enhancing analogs and segments of any of these helper peptides.

The helper peptide may be a universal T-helper epitope. A universal T-helper epitope as used herein refers to a peptide or other immunogenic molecule, or a fragment thereof, that binds to a multiplicity of class II molecules in a manner that activates T cell function in a class II (CD4+ T cells)-restricted manner. An example of a universal T-helper epitope is PADRE (pan-DR epitope) comprising the peptide sequence AKXVAAWTLKAAA (SEQ ID NO: 1). X may be cyclohexylalanyl. PADRE specifically has a CD4+ T-helper epitope, that is, it stimulates induction of a PADRE-specific CD4+ T-helper response. Another example is of a universal T-helper epitope is a non-natural Pan-DR T helper cell epitopes (PADRE). PADRE is a preferred helper peptide particularly suitable for the immunogenic composition disclosed herein.

Tetanus toxoid has other T-helper epitopes that work in the similar manner as PADRE. Tetanus and diphtheria toxins have universal epitopes for human CD4+ cells (Diethelm-Okita 2000). The helper peptide used in the immunogenic composition disclosed herein may be a tetanus toxoid peptide such as F21E comprising the peptide sequence FNNFTVSFWLRVPKVSASHLE (amino acids 947-967; SEQ ID NO: 2).

The immunogenic composition disclosed herein may comprise about 1 or more helper peptides. For example the immunogenic composition may comprise about 1, about 2, about 3, about 4, about 5, about 6, about 7, about 8, about, about 10, about 11, about 12, about 13, about 14, about 15, about 16, about 17, about 18, about 19, or about 20 helper peptides. Typically, it is preferred that an immunogenic composition comprising about 19 tumor-specific neoantigen long and/or short peptides comprises at least about 1 helper peptide.

### d. Tumor-Specific Frameshift Peptide

The immunogenic composition described herein can further comprise a tumor-specific frameshift peptide. Without being bound by theory or mechanism, it is believed that tumor-specific frameshift peptides are highly immunogenic and can lead to enhanced responses to immunogenic compositions (i.e., vaccines).

The tumor-specific frameshift peptide can be of any length. For example, the tumor-specific frameshift peptide may correspond to a tumor-specific neoantigen long peptide or a tumor-specific neoantigen short peptide. The tumor-specific frameshift peptide may be from about 2 amino acids in length to about 100 amino acids in length. Typically, the tumor-specific frameshift peptide maybe be from about 2 amino acids in length to about 30 amino acids in length. The tumor-specific frameshift peptide may be about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or more amino acids in length.

The tumor-specific frameshift peptide may be modified by the addition or deletion of amino acids compared to the tumor-specific frameshift peptide so that the particular peptide is more suitable for inclusion in the immunogenic composition.

The immunogenic composition disclosed herein may comprise about 1 or more tumor-specific frameshift peptides. For example the immunogenic composition may comprise about 1, about 2, about 3, about 4, about 5, about 6, about 7, about 8, about, about 10, about 11, about 12, about 13, about 14, about 15, about 16, about 17, about 18, about 19, or about 20 tumor-specific peptides. Typically, it is preferred that an immunogenic composition comprising about 19 tumor-specific neoantigen long and/or short peptides comprises at least about 2 tumor-specific frameshift peptides.

The tumor-specific frameshift peptides can be included in one or more of the peptide pools.

### e. Adjuvants

The immunogenic composition described herein further comprises an adjuvant. Adjuvants are any substance whose admixture into an immunogenic composition increases, or otherwise enhances and/or boosts, the immune response to a tumor-specific neoantigen, but when the substance is administered alone does not generate an immune response to a tumor-specific neoantigen. The adjuvant preferably generates an immune response to the neoantigen and does not produce an allergy or other adverse reaction. It is contemplated herein that the immunogenic composition can be administered before, together, concomitantly with, or after administration of the immunogenic composition.

Adjuvants can enhance an immune response by several mechanisms including, e.g., activation of Antigen Presenting Cells (APC) like dendritic cells, lymphocyte recruitment, stimulation of B and/or T cells, and stimulation of macrophages. When an immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see, GB 2220211), MF59 (Novartis), AS03 (Glaxo SmithKline), AS04 (Glaxo SmithKline), polysorbate 80 (Tween 80; ICL Americas, Inc.), imidazopyridine compounds (see, International Application No. PCT/US2007/064857, published as International Publication No. WO2007/109812), imidazoquinoxaline compounds (see, International Application No. PCT/US2007/064858, published as International Publication No. WO2007/109813) and saponins, such as QS21 (see, Kensil et al, in Vaccine Design: The Subunit and Adjuvant Approach (eds. Powell & Newman, Plenum Press, NY, 1995); U.S. Pat. No. 5,057,540). In some embodiments, the adjuvant is Freund’s adjuvant (complete or incomplete). Other adjuvants are oil in water emulsions (such as squalene or peanut oil), optionally in combination with immune stimulants, such as monophosphoryl lipid A (see, Stoute et al, N. Engl. J. Med. 336, 86-91 (1997)).

CpG immunostimulatory oligonucleotides have also been reported to enhance the effects of adjuvants in a vaccine setting. Other TLR binding molecules such as RNA binding TLR3, TLR 7, TLR 8, TLR13 and DNA binding TLR 9 may also be used.

Other examples of useful adjuvants include, but are not limited to, poly- ICLC (polyinosinic and polycytidylic acid, stabilized with poly-l-lysine and carboxymethylcellulose), 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel.RTM, vector system, PLGA microparticles, resiquimod, SRL172, Virosomes and other Virus-like particles, VEGF trap, R848, beta-glucan, Pam3Cys, Aquila’s QS21 stimulon, vadimezan, and AsA404 (DMXAA).

### f. Other Components of the Immunogenic Composition

The immunogenic composition described herein can further comprise a pharmaceutically acceptable carrier.

Suspensions or dispersions of one or more tumor-specific neoantigens, especially isotonic aqueous suspensions, dispersions, or amphiphilic solvents can be used. The immunogenic compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use. For injection, the vaccine or immunogenic preparations may be formulated in aqueous solutions, preferably in physiologically compatible buffers such as Hanks’s solution, Ringer’s solution, injectable glucose solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

An excipient can be present independently of an adjuvant. The function of an excipient can be, for example, to increase solubility of vaccine peptides, to increase the molecular weight of the immunogenic composition, to increase activity or immunogenicity, to confer stability, to increase the biological activity, or to increase serum-half life. An excipient can also be used to aid presentation of the one or more tumor-specific neoantigens to T-cells (e.g., CD 4+ or CD8+ T-cells). The excipient can be a carrier protein such as, but not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. Alternatively, the carrier can be dextran, for example sepharose.

Cytotoxic T-cells recognizes an antigen in the form of a peptide bound to an MHC molecule, rather than the intact foreign antigen itself. The MHC molecule itself is located at the cell surface of an antigen presenting cell. Thus, an activation of cytotoxic T-cells is possible if a trimeric complex of peptide antigen, MHC molecule, and antigen-presenting cell (APC) is present. It may enhance the immune response if not only the one or more tumor-specific antigens are used for activation of cytotoxic T-cells, but if additional APCs with the respective MHC molecule are added. Therefore, in some embodiments an immunogenic composition additionally contains at least one APC.

The immunogenic composition can comprise an acceptable carrier (e.g., an aqueous carrier). A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The immunogenic composition disclosed herein, may further comprise one or more emulsifiers. The emulsifier may be a pure emulsifying agent or a mixture of emulsifying agents. The emulsifier(s) should be pharmaceutically and/or immunologically acceptable. An emulsifier may be used to assist in stabilizing an amphiphile, mixture of amphiphile and antigen, or the mixture of amphiphile, antigen and other vaccine components (e.g. adjuvant, helper peptide or tumor-specific frameshift peptide) when the amphiphile or mixtures are resuspended into the hydrophobic carrier. The use of an emulsifier may, for example, promote more even distribution of the amphiphile or mixture in the hydrophobic carrier.

The emulsifier may be amphipathic and therefore, the emulsifier may include a broad range of compounds. The emulsifier may be a surfactant, such as for example, a non-ionic surfactant. Examples of emulsifiers which may be used include polysorbates, which are oily liquids derived from polyethylene glycolyated sorbital, and sorbitan esters. Polysorbates may include, for example, sorbitan monooleate. Typical emulsifiers are well-known in the art and include, without limitation, mannide oleate (Arlacel™A), lecithin, Tween™80, Spans™20, 80, 83 and 85.

Neoantigens can also be administered via liposomes, which target them to a particular cell tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the neoantigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired neoantigen can be directed to the site of lymphoid cells, where the liposomes then deliver the selected immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., An. Rev. Biophys. Bioeng. 9;467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

An alternative method for targeting immune cells, components of the immunogenic composition, such as an antigen (i.e., tumor-specific neoantigen), ligand, or adjuvant (e.g., TLR) can be incorporated into an poly(lactic-co-glycolic) microspheres. The poly(lactic-co-glycolic) microspheres can entrap components of the immunogenic composition as a phago-endosomal delivery device.

For therapeutic or immunization purposes, nucleic acids encoding a tumor-specific neoantigen described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990), as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors, linearized or circular DNA or RNA, with or without electroporation. The nucleic acids can also be delivered complexed to cationic compounds, such as cationic lipids.

Also disclosed herein is a method of manufacturing an immunogenic composition comprising one or more tumor-specific neoantigens selected by performing the steps of the methods disclosed herein. An immunogenic composition as described herein can be manufactured using methods known in the art. For example, a method of producing a tumor-specific neoantigen or a vector (e.g., a vector including at least one sequence encoding one or more tumor-specific neoantigens) disclosed herein can include culturing a host cell under conditions suitable for expressing the neoantigen or vector, wherein the host cell comprises at least one polynucleotide encoding the neoantigen or vector, and purifying the neoantigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes one or more tumor-specific neoantigens or vector disclosed herein. In certain embodiments the isolated polynucleotide can be cDNA.

### B. Methods of Treating

This disclosure also relates to methods of treating cancer in a subject in need thereof comprising administering the personalized immunogenic composition described herein.

The cancer can be any solid tumor or any hematological tumor. The methods disclosed herein are preferably suited for solid tumors. The tumor can be a primary tumor (e.g., a tumor that is at the original site where the tumor first arose). Solid tumors can include, but are not limited to, breast cancer tumors, ovarian cancer tumors, prostate cancer tumors, lung cancer tumors, kidney cancer tumors, gastric cancer tumors, testicular cancer tumors, head and neck cancer tumors, pancreatic cancer tumors, brain cancer tumors, and melanoma tumors. Hematological tumors can include, but are not limited to, tumors from lymphomas (e.g., B cell lymphomas) and leukemias (e.g., acute myelogenous leukemia, chronic myelogenous leukemia, chronic lymphocytic leukemia, and T cell lymphocytic leukemia).

The methods disclosed herein can be used for any suitable cancerous tumor, including hematological malignancy, solid tumors, sarcomas, carcinomas, and other solid and non-solid tumors. Illustrative suitable cancers include, for example, acute lymphoblastic leukemia (ALL), acute myeloid leukemia (AML), adrenocortical carcinoma, anal cancer, appendiceal cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. Preferably, the cancer is melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Melanoma is of particular interest. Breast cancer, lung cancer, and bladder cancer are also of particular interest.

The methods disclosed herein can be used for any suitable cancerous tumor, including hematological malignancy, solid tumors, sarcomas, carcinomas, and other solid and non-solid tumors. Illustrative suitable cancers include, for example, acute lymphoblastic leukemia (ALL), acute myeloid leukemia (AML), adrenocortical carcinoma, anal cancer, appendiceal cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. Preferably, the cancer is melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Melanoma is of particular interest. Breast cancer, lung cancer, and bladder cancer are also of particular interest.

Immunogenic compositions stimulate a subject’s immune system, especially the response of specific CD8+ T cells or CD4+ T cells. Interferon gamma produced by CD8+ and T helper CD4+ cells regulate the expression of PD-L1. PD-L1 expression in tumor cells is upregulated when attacked by T cells. Therefore, tumor vaccines may induce the production of specific T cells and simultaneously upregulate the expression of PD-L1, which may limit the efficacy of the immunogenic composition. In addition, while the immune system is activated, the expression of T cell surface reporter CTLA-4 is correspondingly increased, which binds with the ligand B7-1/B7-2 on antigen-presenting cells and plays an immunosuppressant effect. Thus, in some instances, the subject may further be administered an anti-immunosuppressive or immunostimulatory, such as a checkpoint inhibitor. Checkpoint inhibitors can include, but are not limited to, anti-CTL4-A antibodies, anti-PD-1 antibodies and anti-PD-L1 antibodies, inhibitors of the Lag3 pathway, the Tim3 pathway, the ICOS pathway, the OX-40 pathway, the GITR pathway, or the 4-1BB pathway. These checkpoint inhibitors bind to the immune checkpoint proteins of T cells to remove the inhibition of T cell function by tumor cells. Blockade of CTLA-4 or PD-L1 by antibodies can enhance the immune response to cancerous cells in the patient. CTLA-4 has been shown effective when following a vaccination protocol.

The immunogenic composition described herein can be administered to a subject that has been diagnosed with cancer, is already suffering from cancer, has recurrent cancer (i.e., relapse), or is at risk of developing cancer. The immunogenic composition described herein can be administered to a subject that is resistant to other forms of cancer treatment (e.g., chemotherapy, immunotherapy, or radiation). The immunogenic composition described herein can be administered to the subject prior to, in conjunctions, or after other standard of care cancer therapies (e.g., surgery, chemotherapy, immunotherapy, or radiation). The immunogenic composition described herein can be administered to the subject concurrently, after, or in combination to other standard of care cancer therapies (e.g., chemotherapy, immunotherapy, or radiation).

The subject can be a human, dog, cat, horse, or any animal for which a tumor specific response is desired.

The immunogenic composition described herein can be administered to the subject in an amount sufficient to elicit an immune response to the tumor-specific neoantigen and to destroy, or at least partially arrest, symptoms and/or complications. In embodiments, the immunogenic composition can provide a long-lasting immune response. A long-lasting immune response can be established by administering a boosting dose of the immunogenic composition to the subject. The immune response to the immunogenic composition can be extended by administering to the subject a boosting dose. In embodiments, at least one, at least two, at least three or more boosting doses can be administered to abate the cancer. A first boosting dose may increase the immune response by at least 50%, at least 100%, at least 200%, at least 300%, at least 400%, at least 500%, or at least 1000%. A second boosting dose may increase the immune response by at least 50%, at least 100%, at least 200%, at least 300%, at least 400%, at least 500%, or at least 1000%. A third boosting dose may increase the immune response by at least 50%, at least 100%, at least 200%, at least 300%, at least 400%, at least 500%, or at least 1000%.

An amount adequate to elicit an immune response is defined as a “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that immunogenic compositions can generally be employed in serious disease states, that is, life-threatening or potentially life-threatening situations, especially when the cancer has metastasized. In such cases, in view of the minimization of extraneous substances and the relative nontoxic nature of a neoantigen, it is possible and can be felt desirable by the treating physician to administer substantial excesses of these immunogenic compositions.

The immunogenic compositions provided herein can be administered to the subject by, including but not limited to, oral, intradermal, intrathecal, intratumoral, intramuscular, intraperitoneal, intravenous, topical, subcutaneous, percutaneous, intranasal and inhalation routes, and via scarification (scratching through the top layers of skin, e.g., using a bifurcated needle). The immunogenic composition can be administered at the tumor site to induce a local immune response to the tumor. The preferred administration route, is by intrathecal or intramuscular injection.

The dosage of the immunogenic composition may depend upon the type of composition and upon the subject’s age, weight, body surface area, individual condition, the individual pharmacokinetic data, and the mode of administration.

The subject may be administered one or more doses of the immunogenic composition. In some instances, the immunogenic composition is administered as 1 dose, 2 doses, 3 doses, 4 doses, 5 doses, 6 doses, 7 doses, 8 doses, 9 doses, 10 doses, or more. It is preferable, that the subject is administered 6 doses of the immunogenic composition. While each dose can be administered at the same time, it is preferable that each dose is administered at different times. Each dose can be administered at any suitable interval. Each dose may be administered about 2 day, about 5 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or long after administration of the prior dose. The interval between each dose of the immunogenic composition may be the same time interval or at different time intervals. Each dose is preferably administered at least about 1 week to about 4 weeks after administration of the prior dose of the immunogenic composition. Typically, each dose is administered about 4 weeks after the prior dose of the immunogenic composition.

Generally, it is preferable that the immunogenic composition is administered as two or more peptide pools at each dose. The immunogenic composition can be administered as about 2, about 3, about 4, or about 5 peptide pools at each dose. Preferably, 4 peptide pools are administered at each dose. In some instances, the immunogenic composition is administered as 4 peptide pools at 6 doses.

The subject can be administered each peptide pool in one or more extremities of the subject. The subject may be administered each peptide pool in about 1 to about 4 extremities of the subject. Each peptide pool may be administered on the same extremity of the subject. Each peptide pool may be administered at a different extremity of the subject. It is preferred that each peptide pool is administered at a different extremity of the subject.

Each peptide pool can be administered on the same extremity of the subject at one or more doses. For example, a peptide pool administered to the left arm at each dose. Alternatively, each peptide pool can be administered on a different extremity of the subject at one or more doses. For example, a peptide pool can be administered to the left arm at one dose, the right arm at a second dose, and the right left at a third dose. It is preferable, that each peptide pool is administered on the same extremity of the subject at each dose.

The method can further comprise administering an adjuvant between each dose of the immunogenic composition. The purpose of the adjuvant is to allow for vaccine induced T cell infiltration into tumors, and to support the vaccine induced immune response. The adjuvant may be administered one or more times between each dose of the immunogenic composition. In some instances, an adjuvant may be administered about 1 time, 2 times, 3 times, 4 times, 5 times, 6 times, 7 times, 8 times, 9 times, 10 times, or more between each dose of the immunogenic composition. Typically, the adjuvant is preferably administered weekly between each dose of the immunogenic composition.

The subject may be administered an adjuvant prior to initiation of treatment with the immunogenic composition. The adjuvant may be administered about 1 week, about 2 weeks, about 3 weeks, about 4 weeks or more prior to initiation of the immunogenic composition.

The immunogenic composition may comprise about 10 to about 500 µg of each tumor-specific neoantigen long and/or short peptide per immunogenic composition. The immunogenic composition may comprise about 10 µg, about 20 µg, about 30 µg, about 40 µg, about 50 µg, about 60 µg, about 70 µg, about 80 µg, about 90 µg, about 100 µg, about 110 µg, about 120 µg, 130 µg, 140 µg, about 150 µg, about 160 µg, about 170 µg, about 180 µg, about 190 µg, about 200 µg, about 210 µg, about 220 µg, about 230 µg, about 240 µg, about 250 µg, about 260 µg, about 270 µg, about 280 µg, about 290 µg, about 300 µg, about 310 µg, about 320 µg, about 330 µg, about 340 µg, about 350 µg, about 360 µg, about 370 µg, about 380 µg, about 390 µg, about 400 µg, about 410 µg, about 420 µg, about 430 µg, about 440 µg, about 450 µg, about 460 µg, about 470 µg, about 480 µg, about 490 µg, about 500 µg of each tumor-specific neoantigen long and/or short peptide per immunogenic composition. Typically, the immunogenic composition comprises about 300 µg tumor-specific neoantigen long and/or short peptide per immunogenic composition.

Each peptide pool may be admixed with up to about 900 µg of an adjuvant. For instance, each peptide pool may be admixed with about 10 µg, about 20 µg, about 30 µg, about 40 µg, about 50 µg, about 60 µg, about 70 µg, about 80 µg, about 90 µg, about 100 µg, about 110 µg, about 120 µg, 130 µg, 140 µg, about 150 µg, about 160 µg, about 170 µg, about 180 µg, about 190 µg, about 200 µg, about 210 µg, about 220 µg, about 230 µg, about 240 µg, about 250 µg, about 260 µg, about 270 µg, about 280 µg, about 290 µg, about 300 µg, about 310 µg, about 320 µg, about 330 µg, about 340 µg, about 350 µg, about 360 µg, about 370 µg, about 380 µg, about 390 µg, about 400 µg, about 410 µg, about 420 µg, about 430 µg, about 440 µg, about 450 µg, about 460 µg, about 470 µg, about 480 µg, about 490 µg, about 500 µg, about 550 µg, about 600 µg, about 650 µg, about 700 µg, about 750 µg, about 800 µg, about 850 µg, or about 900 µg of an adjuvant.

The immunogenic composition described herein can be administered to the subject alone or in combination with other therapeutic agents. The therapeutic agent can be, for example, a chemotherapeutic agent, hormone-modulators, signaling cascade inhibitors, radiation, or immunotherapy. Any suitable therapeutic treatment for a particular cancer can be administered. Exemplary chemotherapeutic agents include, but are not limited to aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. The subject may be administered a small molecule, or targeted therapy (e.g. kinase inhibitor). The subject may be further administered an anti-CTLA antibody or anti-PD-1 antibody or anti-PD-Ll antibody. Blockade of CTLA-4 or PD-L1 by antibodies can enhance the immune response to cancerous cells in the patient.

### 7. EXAMPLES

The following examples serve to illustrate, and in no way, limit the present disclosure.

### Example 1

### 1. Personalized Vaccine

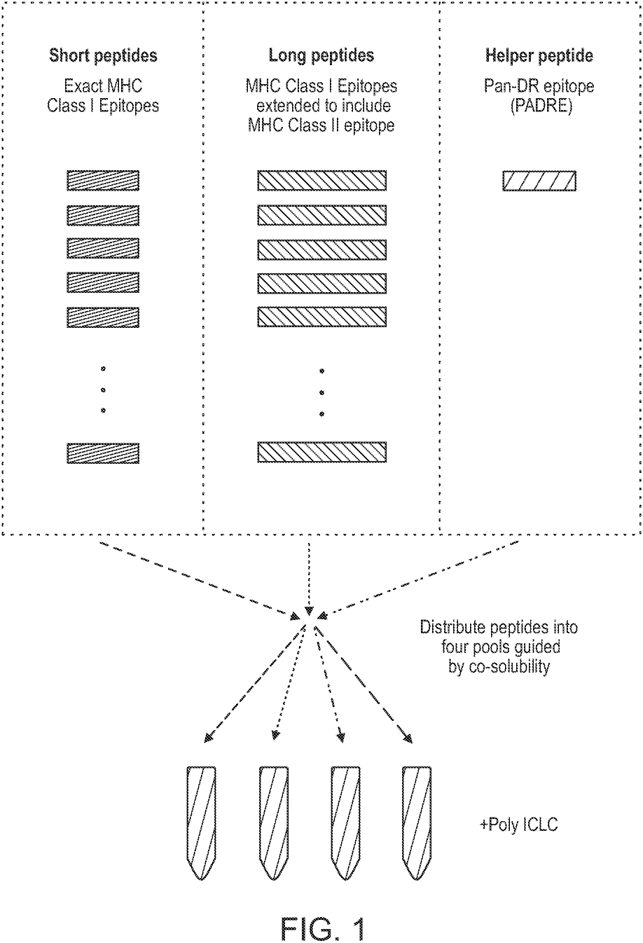

Each personalized vaccine product will be composed of up to four patient-specific, non-complexed peptide-pools of up to five peptides each, combined at the time of administration with a ready-to-use adjuvant Poly ICLC (Hiltonol® (Oncovir, Inc., Washington, DC)). To determine the components of individualized peptide pools, as well as the number of pools, a tissue sample from each patient will undergo DNA and RNA sequencing, in addition to a patient blood sample for HLA typing. The resulting data will be analyzed using multiple bioinformatics algorithms to identify a patient-specific and unique set of neoantigenic peptides with a likelihood of targeting activity toward the patient’s disease. Up to four patient-specific peptide pools will be manufactured to include ≤5 peptides each, along with one pan-DR CD4-helper epitope (PADRE) (FIG. 1).

The composition (which peptides will be combined into which pool) of each vaccine pool is guided by information about the solubility of the peptides and predicted immunogenicity/recipient immunological responsiveness. Specifically, physico-chemical properties of peptides like the fraction of hydrophobic amino acids determine which peptides are best co-soluble. The manufacturer will be provided with the predicted immunogenicity from the computational pipeline and machine learning models. The manufacturer will attempt to compose peptide pools with similar average immunogenicity of all peptides included.

Essentially, each patient specific vaccine will consist of less than 19 peptides representing naturally presented MHC Class I epitopes, or elongated versions thereof, including at least one neoantigenic tumor specific mutation. These peptides allow for presentation on antigen presenting cells, which are expected to induce a T-cell mediated anti-tumor immune reaction. To ensure co-solubility of the vaccine peptide, they will be divided into up to four peptide pools for individual administration. During formulation, a vaccine adjuvant (here Poly ICLC, Hiltonol®) will be added to each peptide pool to support the generation of a desired T-cell response. Further, PADRE, a pan-DR helper epitope will be included in one of these peptide pool vaccines with the goal of inducing a CD4 T-cell mediated “helper” response, thereby supporting CD8 T-cell priming and proliferation.

The peptides will each be of ≤30 amino acids (AA) length, all composed of naturally occurring L-amino acids without modifications. The peptide sequences will differ from patient normal protein sequences as they all incorporate at least one mutation (non-synonymous, or due to indels) found in the patient’s tumor. The PADRE peptide has been described previously (AKFVAAWTLKAAA (SEQ ID NO: 3)) and consists of naturally occurring L-amino acids. Except for the PADRE peptide, the peptide sequences contained within the product vary between patients.

Poly ICLC (also known as Hiltonol®) will be used as the adjuvant component of the personalized vaccine. Additionally, Poly ICLC is planned to be injected in between the six planned vaccination visits to support tumor-homing of neo-antigen specific T cells induced by the vaccine, and to reduce the immune-suppressive tumor microenvironment.

Polyinosinic-Polycytidylic acid stabilized with polylysine and carboxymethylcellulose, is a stabilized double-stranded RNA (dsRNA) that was used as an interferon (IFN) inducer at high doses (up to 300 mcg/kg IV) in short-term cancer trials some years ago. However, lower dose (10 to 50 mcg/kg) Poly-ICLC has been shown to induce a broader host defense, a potent adjuvant effect, and a specific anti-tumor and antiviral effect. Poly-ICLC also has been found to preferentially decrease tumor protein synthesis as well as cell proliferation in vivo.

### 2. Dosage Form and Administration

In the proposed phase I clinical trial, the tumor-specific neoantigens together with Poly ICLC will be administered to the study participants. Common to both indication groups, a single dose of vaccine, and of Poly ICLC will be given as provided in Table 1 below.

Table 1.

TABLE 1

Vaccine dosing

| Dose level | Vaccine Dose |
|---|---|
| 1 | Up to four pools with five or less peptides each (300 µg/peptide/dose) admixed with 500 mcg Poly ICLC in a total volume of 1 mL per pool |
| Dose level | Poly ICLC Dose |
| 1 | Single IM injection, 1.8 mg in a total volume of 1 mL |

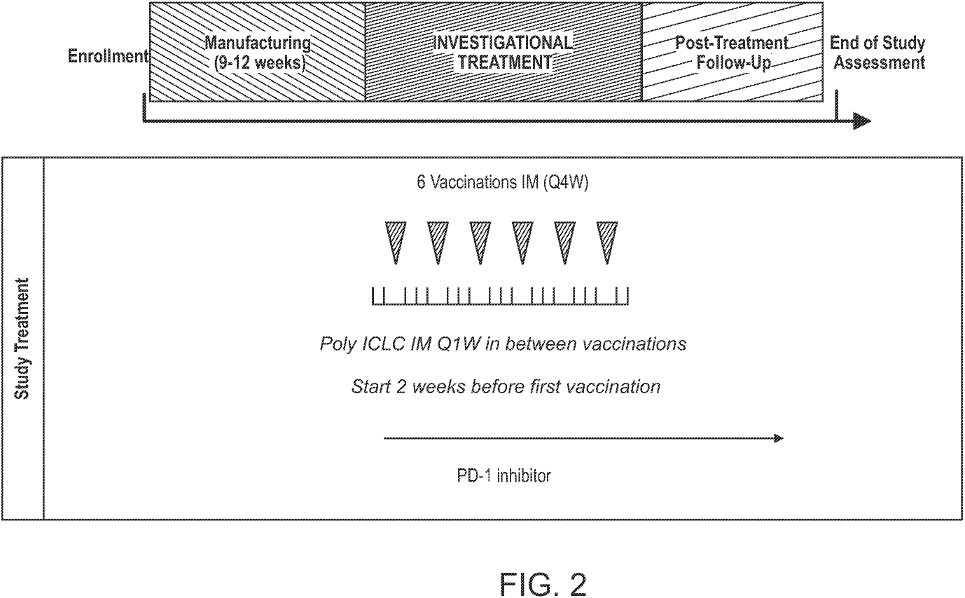

Six doses of the four vaccine pools (peptide pool admixed with Poly ICLC adjuvant) will be administered IM once every four weeks. These vaccinations are flanked with Poly ICLC IM injection once every week except for weeks in which vaccine is given. The purpose of IM Poly ICLC injections is to allow for vaccine induced T cell infiltration into the tumors, and to support the vaccine induced Th1 immune response. The treatment regimen is shown in FIG. 2.

### 3. Vaccine Formulation

The vaccine will undergo final formulation “at-bedside” at the clinical site so as to be uniquely tailored to each patient’s tumor antigen profile and predicted immune responsiveness. Peptide pools will be diluted to a desired concentration of 300 µg/peptide in an adjuvant solution (500 µg Poly ICLC) in a total injectable solution of 1 mL per pool. The procedure will follow standard procedure for vaccine preparation and administration, with the requirement of an additional step of filtering the vaccine peptide pool product through a sterile particle filter prior to mixing with the adjuvant.

The choice of vaccine peptides to be formulated into vaccine pools will be determined by their predicted immunogenicity, and physicochemical properties governing solubility and manufacturability. Individual peptides to be formulated will be identified collaboratively by the study group and the peptide manufacturer, aiming to incorporate the highest number of immunogenic peptides (≤20, optionally including PADRE).

In order to dissolve as many peptides ranked as highly immunogenic by our vaccine peptide prediction algorithm as possible, DMSO will be used. DMSO in low concentrations (approximately 4% v/v) is used as excipient in various approved drugs and has been shown to be safe when used as cryoprotectant in stem cell infusions.

### Example 2: Phase I Clinical Trial for Treating Subjects With Stage IIIC-IV Melanoma or Hormone Receptor Positive Her2 Negative Breast Cancer

A phase I clinical trial is used to study the safety of a personalized neo-antigen peptide vaccine in treating about twenty subjects with metastatic or refractory stage IIIC-IV melanoma or hormone receptor positive Her2 negative breast cancer. Administering personalized neo-antigen peptide vaccine together with Th1 polarizing adjuvant poly ICLC may induce a polyclonal, poly-epitope, cytolytic T cell immunity against the patient’s tumor.

The specific diseases being studied are anatomic stage IV breast cancer AJCC v8, clinical stage III cutaneous melanoma AJCC v8, clinical stage IV cutaneous melanoma AJCC v8, hormone receptor-positive breast carcinoma, locally advanced cutaneous melanoma, metastatic acral lentiginous melanoma, metastatic conjunctival melanoma, metastatic cutaneous melanoma, metastatic Her2-negative breast carcinoma, metastatic mucosal melanoma, pathologic stage IIIC cutaneous melanoma AJCC v8, pathologic stage IIID cutaneous melanoma AJCC v8, pathologic stage IV cutaneous melanoma AJCC v8, prognostic stage IV breast cancer AJCC v8, recurrent acral lentiginous melanoma, recurrent cutaneous melanoma, recurrent mucosal melanoma, refractory Her2-negative breast carcinoma, unresectable acral lentiginous melanoma, unresectable cutaneous melanoma, and unresectable mucosal melanoma.

### 1. Outline

Subjects receive a personalized neo-antigen peptide vaccine by intramuscular injection once every four weeks. Subjects receive poly ICLC (Hiltonol®, poly I:poly C with poly-L-lysine stabilizer, polyinosinic-polycytidylic Acid Stabilized with polylysine and carboxymethylcellulose, polyriboinosinic-polyribocytidylic Acid-polylysine carboxymethylcellulose, stabilized polyriboinosinic/polyribocytidylic acid) intramuscularly once weekly in weeks when no vaccine is given. The vaccine is first administered two weeks after starting poly ICLC, and once every four weeks thereafter. Also two weeks after starting poly ICLC, nivolumab (BMS-936558, CMAB819, MDX-1106, NIVO, nivolumab biosimilar CMAB819, ONO-4538, Opdivo® (Bristol Myers Squibb, New York, NY)) is administered intravenously every two or four weeks. The duration of vaccine treatment is 25 weeks, unless the subject’s disease progresses or treatment brings about unacceptable toxicity.

After the completion of vaccine treatment, nivolumab is administered to the subject every two or four weeks for up to twelve months, in the absence of disease progression or unacceptable toxicity.

After completion of study treatment, subjects are followed up at 24, 36, and 48 weeks.

### 2. Outcome Measures

The primary outcome measure is incidence of adverse events in the 1-year period after the first dose of the personalized neo-antigen vaccine. Adverse events are assessed by Common Terminology Criteria for Adverse Events version 5.0.

Secondary outcome measures are the number of formulated and administered personalized neo-antigen vaccines after 48 weeks; the number of personalized neo-antigen vaccines formulated with at least five (5) vaccine peptides, also after 48 weeks; the number of personalized neo-antigen vaccines formulated in less than 16 weeks after the screening visit biopsy; best overall response assessed by immune-related Response Evaluation Criteria in Solid Tumors criteria in the 1-year period after the first dose of the personalized neo-antigen vaccine; and progression-free survival in the 1-year period after the first dose of the personalized neo-antigen vaccine.

### 3. Eligibility Criteria

The study is open to adults 18 years and older, of any sex. Healthy volunteers are not accepted.

### 8. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

1. An immunogenic composition comprising:
(a) a plurality of tumor-specific neoantigen long peptides;
(b) a plurality of tumor-specific neoantigen short peptides; and
(c) an adjuvant.

2. The immunogenic composition of claim 1, wherein the immunogenic composition comprises up to about 50 tumor-specific neoantigen long peptides and/or short peptides.

3-9. (canceled)

10. The immunogenic composition of claim 1, wherein each of the tumor-specific neoantigen long peptides in the immunogenic composition are different and/or each of the tumor-specific short peptides in the immunogenic composition are different.

11. (canceled)

12. The immunogenic composition of claim 1, wherein the immunogenic composition comprises one or more tumor-specific frameshift peptides.

13. The immunogenic composition of claim 1, wherein the tumor-specific neoantigen long peptides and/or short peptides are divided into two or more peptide pools.

14. (canceled)

15. The immunogenic composition of claim 13 , wherein each peptide pool comprises about 5 or less tumor-specific neoantigen long peptides and/or short peptides.

16. The immunogenic composition of claim 13, wherein one or more peptide pools comprise a helper peptide and/or one or more tumor-specific frameshift peptides.

17-22. (canceled)

23. The immunogenic composition of claim 1, wherein the adjuvant is a Toll-like receptor agonist, a NOD-like receptor agonist, an Mda5 agonist, a RIG-I, PKR agonist, a STING agonist, or other innate immune sensing pathway agonists.

24. The immunogenic composition of claim 1, further comprising a helper peptide, wherein the helper peptide is a pan-DR helper epitope (PADRE), a tetanus helper peptide, a hepatitis B surface antigen helper T-cell epitope, a pertussis toxin helper T-cell epitope, a measles virus F protein helper T-cell epitope, a Chlamydia trachomitis major outer membrane protein helper T-cell epitope, a diphtheria toxin helper T-cell epitope, a Plasmodium falciparum circumsporozoite helper T-cell epitope, a Schistosoma mansoni triose phosphate isomerase helper T-cell epitope, a keyhole limpet hemocyanin, a Plasmodium vivax B cell epitope (PVB), a Escherichia coli TraT helper T-cell epitope, a synthetic T helper epitope, immune-enhancing analogs and segments of any of the foregoing helper peptides.

25. (canceled)

26. The immunogenic composition of claim 13, wherein each of the peptide pools further comprise the adjuvant.

27. (canceled)

28. A pharmaceutical composition comprising the immunogenic composition of claim 1 and a pharmaceutically acceptable carrier.

29. A method of treating cancer, comprising administering to a subject in need thereof a therapeutically effective amount of the immunogenic composition of claim 1.

30. The method of claim 29, wherein the subject is administered at least one or more doses of the immunogenic composition.

31. (canceled)

32. The method of claim 29, wherein the immunogenic composition is administered as one or more peptide pools at each dose.

33-35. (canceled)

36. The method of claim 32, wherein the subject is administered each peptide pool on a different extremity of the subject.

37. The method of claim 32, wherein the subject is administered each peptide pool on the same extremity of the subject at each administration.

38. The method of claim 32, wherein the subject is administered two or more doses of the immunogenic composition, and each dose of the immunogenic composition is administered at least about 1 week to about 4 weeks after administration of the prior dose of the immunogenic composition.

39. The method of claim 30, wherein the subject is administered two or more doses of the immunogenic composition, further comprising administering an adjuvant between each dose of the immunogenic composition.

40. (canceled)

41. The method of claim 39 , wherein the adjuvant is the a Toll-like receptor agonist, a NOD-like receptor agonist, an Mda5 agonist, a RIG-I, PKR agonist, a STING agonist, or other innate immune sensing pathway agonists.

42. The method of claim 29, further comprising administering at least one checkpoint inhibitor.

43. The method of claim 42, wherein the checkpoint inhibitor is an inhibitor of the programmed death-1 (PD-1) pathway, Lag3 pathway, Tim3 pathway, ICOS pathway, OX-40, GITR pathway, or 4-1BB pathway.

44-45. (canceled)

46. The method of claim 29, wherein the cancer is melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, colon cancer, urothelial cancer, or lung cancer.

47-50. (canceled)

51. The method of claim 29, wherein the immunogenic composition is administered by subcutaneous, intramuscular, transcutaneous, intradermal, transdermal, intra-tumoral, into a lymph node, intravenous or intraperitoneal administration.

52. (canceled)


SEQUENCE LISTING (3 sequences)

SEQ ID NO: 1 - protein, 13 aa, Artificial Sequence
  Description of Artificial Sequence: Synthetic peptide
  MOD_RES LOCATION: (3)..(3): Cyclohexylalanyl
AKXVAAWTLKAAA

SEQ ID NO: 2 - protein, 21 aa, Clostridium tetani
FNNFTVSFWLRVPKVSASHLE

SEQ ID NO: 3 - protein, 13 aa, Unknown
  Description of Unknown: PADRE peptide sequence
AKFVAAWTLKAAA